(19) United States
(12) Patent Application Publication
(10) Pub. No.: US 2014/0039460 A1
(43) Pub. Date: Feb. 6, 2014

(54) Transluminal delivery of regenerative cells to body tissue

(71) Applicant: Eckhard Alt, Houston, TX (US)

(72) Inventor: Eckhard Alt, Houston, TX (US)

(73) Assignee: SciCoTec GmbH, Gruenwald (DE)

(21) Appl. No.: 13/986,541

(22) Filed: May 13, 2013

Related U.S. Application Data
(63) Continuation-in-part of application No. 12/255,550, filed on Oct. 21, 2008, now abandoned.
Application No. 12/255,550 is a continuation of application No. 10/955,403, filed on Sep. 30, 2004, now Pat. No. 7,452,532.
Application No. 10/955,403 is a continuation-in-part of application No. 09/968,739, filed on Sep. 30, 2001, now Pat. No. 6,805,860.

(51) Int. Cl. A61M 25/10

(52) U.S. Cl. CPC A61M 25/10
USPC 604/509

(57) ABSTRACT

A method is described for optimizing regenerative cell retention at a tissue site in the body of a subject. In the method, the regenerative cells are delivered to the tissue site through a blood vessel in communication with the site, while blood flow through the vessel is occluded at the site for a prescribed period of sufficient duration to increase pressure at the site for enhanced concentration and retention of the regenerative cells at the tissue site, but of insufficient duration to cause damage to healthy tissue at the site for lack of blood perfusion during the period of occlusion.

### CROSS-REFERENCE TO RELATED APPLICATIONS

This application is a continuation in-part of Ser. No. 12/255,550, filed Oct. 21, 2008, now abandoned (coincident with the filing of this application), which is a continuation of Ser. No. 10/955,403, filed Sep. 30, 2004, which is a continuation-in-part of Ser. No. 09/968,739, filed Sep. 30, 2001, now U.S. Pat. No. 6,805,860, issued Oct. 19, 2004. Applicant claims priority to the parent applications, which are incorporated herein by reference in their entireties.

### FIELD OF THE INVENTION

The present invention relates generally to application of regenerative cells into the body of a patient via the lumen of a vessel or duct of the body, and more particularly in two distinct respects, namely, (i) to increase pressure and thereby, concentration and retention of the regenerative cells within a preselected region or site of tissue in communication with the vessel or duct and to promote migration of the regenerative cells into the tissue thereat, and (ii) ultimately, by virtue of capability of the regenerative cells for differentiation to host tissue in vivo over time, to effect repair, recovery, regeneration or restoration of an organ in a region or at a site having a need for such treatment.

Regenerative cells are of a type or group that possess the capability to repair, renew, regenerate or restore tissue of body parts, i.e., to differentiate to tissue, parenchym and mesenchym, including tissue of organs such as the heart, liver, brain, kidney, and pancreas, as well as of small and large blood vessels, nerves, glands and muscles. Regenerative cells include stem cells derived from a human or animal subject, such as from the umbilical cord or from an individual at any stage of life (i.e., adult stem cells), autologous or allogeneic (allogenic), uncultured or fresh, un- or minimally manipulated or engineered cells. The principles of the invention are applicable to regenerative cells in the broadest sense of that terminology.

### BACKGROUND OF THE INVENTION

Although initial discussion in this specification is directed to application of methods of the invention to the heart, the present invention is not so limited. Rather, it is applicable as well to other organs and body parts, such as the liver, the brain, the kidneys, and the pancreas, for example, and even to related peripheral circulation, muscles, nerves, glands and other tissues, as will become apparent to the reader from the ensuing description.

In principle, the human body has three types of normal (or benign) cells. One type constitutes cells that continuously undergo replication and reproduction, such as dermal cells and epithelial cells of the intestine, for example. These cells, which have a life as short as ten days, are replaced by the same cell type that is continuously replicating. A second type of cell is differentiated in the adult state, but has the potential to undergo replication and the ability to reenter the cell cycle under certain conditions, such as liver cells, for example. The liver has the capacity to regrow and repair itself even if a tumor is excised, and even if a major portion of the liver is removed. The third cell type comprises those cells that stop dividing after they have reached their adult stage, such as neuro cells and myocardial cells, for example.

For the latter type or group, the number of cells in the body is determined shortly after birth. For example, myocardial cells stop dividing at about day ten after delivery, and a fixed number of myocardial cells remains for the rest of life of the human body. Changes in myocardial function occur not by division and new cell growth, but instead often as a result of hypertrophy of the cells.

Although the absence of cell division in myocardial cells is beneficial to prevent the occurrence of tumors—which practically never occur in the heart—it is detrimental with regard to local repair capacities. During the individual's lifetime, myocardial cells are subjected to various causes of damage or disease, which may irreversibly lead to cell necrosis.

The primary reason for cell death in the myocardium is ischemic heart disease—in which the blood supply to the constantly beating heart is compromised through either arteriosclerotic build-up or acute occlusion of a vessel following a thrombus formation, resulting in myocardial infarction (MI). The period of ischemic tolerance of myocardial cells following substantially complete blockage of the blood supply is in a range of three to six hours. Thereafter, the affected myocardial cells undergo necrosis and replacement by scar tissue.

An event of MI leads to irreversible loss of functional cardiac tissue with deterioration of the heart's pumping function and possible death of the individual, absent immediate life-saving intervention. Heart attack remains the leading cause of death throughout the world, except in some third world countries. Occlusion of a coronary vessel causes interruption of the oxygenated blood supply of the dependent capillary system, and, after loss of oxygen within the aforementioned period, myocardial cell death. Fibrotic scarring occurs, and contribution of this part of the heart to the contractile force is lost. The cardiac muscle seeks to compensate for this loss through hypertrophy of the remaining unaffected cardiomyocytes, since the capability of myocardial cells to divide is lost within a short time after mammalian birth.

Other myocardial cell alteration may occur as the result of cardiomyopathy, a primary disease process of heart muscle in absence of a known underlying etiology. Among the types of cardiomyopathy, for example, are endocrine, metabolic (alcohol) or infectious (virus myocarditis) agents that lead to cell death, and consequently reduced myocardial pumping function. An increasing group of patients suffer myocardial damage following certain treatment for cancers as well, such as breast or gastrointestinal or bone marrow cancers, attributable to the toxic effect of the cytostatic agents used in the treatment.

Traditionally, treatment of patients with heart disease has been performed using interventional cardiology—such as PTCA (percutaneous transluminal coronary angioplasty), balloon angioplasty or stent implantation, for example,—or surgical revascularization with bypass operation. Stunned and hibernating myocardial cells, i.e., cells that survive on a low energy level but are not contributing significantly to the myocardial pumping function, may recover improved contractile capability. But no recovery has been attainable for those myocardial cells that are already dead.

The current state of interventional cardiology is one of high standard. Progress in balloon material, guide wires, and guiding catheters, and in experience of the interventional cardiologist, as well as the use of concomitant medication to enhance patient recovery, has greatly improved the practice of cardiology. Nevertheless, an acute myocardial infarction remains an event that, even with presently available optimal treatment, can lead to a loss of from 25 to 100% of the area of the myocardium at risk from vessel blockage. Complete re-canalization is feasible, limited by the ischemic tolerance of the myocardium.

An article published in the New England Journal of Medicine (Schomig A. et al., “Coronary stenting plus platelet glycoprotein IIWIIIa blockade compared with tissue plasminogen activator in acute myocardial infarction,” N Engl J Med 2000; 343:385-391), for which the applicant herein was a clinical investigator, reports on a study of the myocardial salvage following re-canalization in patients with an acute myocardial infarction. The average time until admission to the hospital in these patients was 2.5 hours and complete re-canalization was feasible after 215 minutes, roughly 3.5 hours. Nevertheless, only 57% of the myocardium at risk could be salvaged by re-canalisation through interventional cardiology by means of a balloon and stent. When the group of patients was randomized to the classical thrombolytic therapy (dissolution of blood clots), which was the worldwide standard (with no interventional means), only 26% of the myocardium at risk could be salvaged. This means that despite optimal circumstances more than 40% of the myocardial cells are irreversibly lost.

Recognizing that many patients arrive at the hospital at from 6 to 72 hours after the acute symptoms of vessel blockage by a thrombus, considerably longer than the optimal situationed referred to above, it can be assumed that the average loss of affected myocardial tissue is in a range of from 75 to 90% following an acute MI.

As noted earlier herein, myocardial cells can survive on a lower energy level, referred to as hibernating and stunning myocardium. As the collateral blood flow increases or re-canalization provides new blood supply, those cells can recover their contractile function. The principle of myocardial re-perfusion, limitation of infarct size, reduction of left ventricular dysfunction and their effect on survival were described by Braunwald (Braunwald E. et al., “Myocardial reperfusion, limitation of infarct size, reduction of left ventricular dysfunction, and improved survival: should the paradigm be expanded?,” Circulation 1989; 79:441-4).

Annually, some five million Americans survive an acute myocardial infarction. Clearly then, loss of affected myocardial tissue is a problem of major clinical importance. Restoration of a portion of the consequent loss of pumping function is limited to hypertrophy of the remaining myocardium, and optimal medical treatment by a reduction in pre- and after-load as well as the optimal treatment of the ischemic balance by p-blockers, nitrates, calcium antagonist, and ACE inhibitors.

If it were feasible to replace the damaged myocardial cells by growing new myocardial cells in intimacy therewith, or to repair the damaged tissue cells, so as to recover a substantial portion of the pumping function lost by inactivity of the former, such a technique would have a profoundly favorable impact on the quality of life of affected patients.

The group of William C. Claycomb et al. has been engaged in research on the behavior and the development of myocytes since the early 1970's. In their initial report (Goldstein M. A. et al., “DNA synthesis and mitosis in well-differentiated mammalian cardiocytes,” Science 1974; 183:212-3), they described the incorporation of 3H-Thymidin into the nuclei of heart cells of two days old rats which indicates that neonatal cardiac cells still undergo synthesis of DNA and divide despite the presence of contractile proteins. This phenomenon of cell division ceases at day 17 of the postnatal development. After that time no further division of cardiac cells occurs, either in rats or in humans.

The interest in mammalian cardiomyocytes has led to the development of cultures of adult cardiac muscle cells (Claycomb W. C. et al., “Culture of the terminally differentiated adult cardiac muscle cell: A light and scanning electron microscope study,” Dev Biol 1980; 80:466-482), and ultimately to the generation of a transplantable cardiac tumor-derived transgenic AT 1-cell.

During the 1980's intensive studies were conducted with the characterization of this atrial derived myocyte cell line, which is immortalized by the introduction of the SV40-largeT-oncogene (SV40-T). From this AT-1-cell-group, other adult cardiomyocytes have been derived. These can be passaged indefinitely in culture, can be recovered from a frozen stock, can retain a differentiated cardiomyocyte phenotype, and maintain their contractile activity.

They are described as HL-1-cells. The reader is referred, for example, to Delcarpio J. B. et al., “Morphological characterization of cardiomyocytes isolated from a trans-plantable cardiac tumor derived from transgenic mouse atria (AT-1 cells),” Circ Res 1991; 69(6):1591-1600; Lanson Jr. N. A. et al., “Gene expression and atrial natriuretic factor processing and secretion in cultured AT-1 cardiac myocytes,” Circulation 1992; 85(5):1835-1841; Kline R. P. et al., “Spontaneous activity in transgenic mouse heart: Comparison of primary atrial tumor with cultured AT-1 atrial myocytes,” J Cardiovasc Electrophysiol 1993; 4(6):642-660; Borisov A. B. et al., “Proliferative potential and differentiated characteristics of cultured cardiac muscle cells expressing the SV 40 T oncogene,” Card Growth Reg 1995; 752:80-91; and Claycomb W. C. et al., “HL-1 cells: A cardiac muscle cell line that contracts and retains phenotypic characteristics of the adult cardiomyocyte,” Proc Natl Acad Sci USA 1998; 95:2979-84.

Later, the cardiomyocyte transplantation in a porcine myocardial infarction model was studied intensively in collaboration with the research group of Frank Smart (Watanabe E. et al., “Cardiomyocyte transplantation in a porcine myocardial infarction model,” Cell Transplant 1998; 7(3):239-246). In conjunction with the AT-1 cardiomyocytes, human fetal cardiomyocytes were injected through a syringe and needle into the adult pig heart infarction area.

In summary, these cells showed local growth and survived in the infarction border zone, but could not be found in the core scar tissue of the myocardial infarction. The majority of the implanted cells were replaced with inflammatory cells, suggesting that the immunosuppressant regimen that was concomitantly applied was not sufficient for the grafted cells to survive in the host myocardium. Other factors that may have influenced the result that the transplanted cells were not detected, could possibly be linked to the fact that the cells were grafted 45 days after inducing the infarction.

The inflammatory stimuli for cell growth are significantly reduced in the first two to three weeks of an MI. Also, that transforming-growth-factor-f3 (TGF-(3), fibroblast-growth-factor-2 (FGF-2), platelet-derived-growth-factor (PDGF) and other cytokines, like the interleucin-family, tumor-necrosis-factor-a (TNF-a) and interferon-gamma are strong stimulators of cell proliferation and cell growth. The adjunct therapy with immunosuppression has further reduced this stimuli for cell growth.

Another major factor for the failure of detection of grafted cells in the myocardial scar may be the selection of the infarction model. An artery is occluded and the blood supply has not recovered before grafting. There is no reason to assume that the grafted cells could survive in an ischemic area and grow, any better than the myocytes.

Accordingly, other groups have tried to induce a myocardial angiogenesis by gene-therapy. This was either performed by the administration by fibroblast growth factor H in the presence or absence of heparin (see Watanabe E. et al., “Effect of basic fibroblast growth factor on angiogenesis in the infarcted porcine heart,” Basic Res Cardiol 1998; 93:30-7), or by application of vascular endothelial growth factor (VEGF), a potent mitogen for endothelial cells. VEGF stimulates capillary formation and increases vascular permeability (Lee J. S. et al., “Gene therapy for therapeutic myocardial angiogenesis: A promising synthesis of two emerging technologies,” Nat Med 1998; 4(6):739-42). Still other groups have tried to increase the collateral capillary blood flow by human bone marrow derived angioblasts and have shown an improvement in acute myocardial infarction in rats treated with injections of colony-stimulating-factor-G (CSF-G) mobilized adult human CD-34 cells (Kocher A. A. et al., “Neovascularization of ischemic myocardium by human bone-marrow-derived angioblasts prevents cardiomyocyte apoptosis, reduces re-modeling and improves cardiac function.” Nat Med 2001; 7(4):430-6).

While these approaches certainly have some research merit, their clinical relevance for the majority of patients is not as important, since we have effective means to re-canalize an occluded vessel and provide a blood supply via the natural branching of the coronary arteries, which further subdivides into arterioles and capillaries.

Other attempts to transplant preformed patches also necessitate the growth of the grafted cells in a patch formation and a surgical operation in a patient, which requires opening the thoracic cage.

Considering the complications, the cost and the risk associated with these time consuming procedures, it becomes clear that they offer only limited likelihood for widespread routine application.

Other groups have tried to make use of the precursor cells that are found in the peripheral muscle. Unlike the heart, there is a certain degree of repair in peripheral skeletal muscles, since the peripheral skeletal muscle contains progenitor cells, which have the capability to divide and replace the peripheral muscle. By isolating those cells from a probe of a thigh muscle, the progenitor cells of skeletal muscle have been separated, cultured and re-injected in an animal model (Taylor D. A. et al., “Regenerating functional myocardium: Improved performance after skeletal myoblast transplantation,” Nat Med 1998; 4(8):929-33; Scorsin M. et al., “Comparison of the effects of fetal cardiomyocyte and skeletal myoblast transplantation on postinfarction left ventricular function,” J Thorac Cardiovasc Surg 2000; 119:1169-75), and more recently in some patients also.

The application of these cultured cells has also been attempted by injection with small needles following an opening of the subject's chest and the pericardial sac. While in the model of kryo-infarction, in which only the myocardial cells die but the blood supply through the vascular system is not limited, the injection of autologous skeletal myoblasts improves the myocardial function. The results indicated, however, that the engrafted cells retain skeletal muscle characteristic, which means they cannot contract at the constant fast rate imposed by the surrounding cardiac tissue. In addition, no electrical connection exists between the graft cells and the host tissue, and it is assumed that their contribution to improve contractile performance probably resulted from the mechanical ability of the engrafted contractile tissue to respond to stretch activation by contraction.

Considering the experience with latissimus dorsi muscle grafting—a procedure called dynamic cardiomyoblasty—, the disappointing results with the possible use of skeletal muscle as a myocardial substitute indicate that the long term different muscle characteristics of skeletal muscles do not match the need of a constantly pumping myocardial cell. Therefore, the best these cells might achieve would be to improve the quality of the scar of the ischemic myocardium, but not actively contribute to a contraction of this area in the long term.

### BRIEF SUMMARY OF THE INVENTION

The applicant herein has determined that it is essential to improve the manner in which regenerative cells are applied to a target site of body tissue designated to undergo or be subjected to treatment in an effort to overcome tissue damage at the site. This means a need to achieve appropriate levels of concentration of the regenerative cells at or in the immediate vicinity of the site of interest, and levels of retention (numbers) of the regenerative cells by tissue at the site. Additionally, it is necessary that the regenerative cells be derived from a source that practically assures their secure attachment and ultimate differentiation to assume the same characteristics as the host tissue at the site selected for their delivery. Lack of the characteristics of the host tissue cells has plagued at least some of the efforts in the prior research activity cited above.

As stated earlier herein, a principal aspect of the present invention is to the transluminally deliver regenerative cells to a target site of body tissue in communication with a blood vessel or duct through which the cells are injected, in levels of concentration and retention that markedly improve the likelihood of successful treatment. The purpose of the treatment may not necessarily target organ recovery, regeneration, renewal or repair, or anything other than delivering applicable regenerative cells in sufficient numbers prescribed by the attending physician or surgeon as having the capacity to enhance the rejuvenation or strengthening of performance of the selected site of the tissue including tissue underlying the vessel's surface. The essence of the invention resides in optimization of delivery to a targeted site of tissue damage, of regenerative cells having the capacity to differentiate themselves in vivo to host tissue at the site, in a manner that the delivery results in levels of concentration and retention of the delivered cells heretofore unattainable, and further enables migration of the regenerative cells to the damaged tissue. Although the methods of the invention are applicable to delivery of regenerative cells that may be derived from any suitable source, the cells used in the exemplary methods of transluminal application to be described are autologous adult stem cells, that is, regenerative cells harvested from beyond the earliest stage of life of the cell donor in which they are to be used. Preferably, the delivery procedure is commenced shortly after these stem cells are harvested. Sources for harvesting of the stem cells include the subject's bone marrow, or adipose (fat) tissue, or lipoaspirate (withdrawn from fatty tissue).

According to an important aspect of the invention, the vessel chosen for the transluminal application of the regenerative cells is temporarily blocked during a portion of the procedure. In the case of a blood vessel, the blockage is limited by the maximum permissible period of time before lack of oxygenated blood is likely to cause tissue cell death. That period may depend on the particular organ or other body part nourished by the blood flow in the vessel. For example, comparatively the heart can tolerate a greater period in which blood flow is blocked than the period of blockage tolerated by the brain. The purpose of the blockage with concomitant delivery of the regenerative cells is to facilitate an increase in pressure in the vessel bed at the target site distal to the location of occlusion so as to prevent washout of the cells by blocking them from flowing backward during their transluminal application. The applicant has found by experimental results that this procedure achieves significantly higher levels of concentration and retention of regenerative cells at the selected site, and cell migration to overcome the endothelial barrier at the site. At the end of the prescribed blockage period, the vessel's lumen is opened to allow normal blood flow therethrough.

It is therefore a principal aim of the invention to provide a method for improved delivery of regenerative cells to a site in the body undergoing treatment in order to achieve an increased retention of the cells at the target site.

More specifically, the invention aims to provide concentration and retention of regenerative cells at a selected site of body tissue under treatment, at levels that had heretofore not been achievable.

Preferably, occlusion of the vessel through which the regenerative cell delivery is made is performed by inflating the balloon of a balloon catheter that has been advanced through the vessel's lumen, preferably over a guidewire or guiding catheter, to place the distal end of the balloon catheter at or in close proximity to the selected (i.e., target) site of the body tissue under treatment. The inflation is initiated at or immediately prior to the commencement of injection of the regenerative cells (through a central lumen of the catheter different from the inflation lumen), so as to block perfusion of blood at the location of the inflated balloon, which is also the outlet for cell delivery to the site. Cell delivery is performed at a predetermined injection rate.

Occlusion of the vessel and consequent lack of blood flow at the site during application of the regenerative cells serves to facilitate an increase in the pressure downstream at the target site, to prevent backflow-related washout of the injected cells and to overcome the endothelial barrier that exists by virtue of an existing layer of flat cells (the endothelium) in the wall of the blood vessel and the target site. Backflow of the cells is prevented by forcing them, with this increase in the pressure during injection, to go forward. Since the downstream capillary network has only a limited capacity to allow the cells to pass through in a given time, a temporary congestion of cells in their suspension fluid occurs in the vascular space between the balloon occlusion and the distal capillary bed. The increased pressure in this vascular segment forces the cells into the surrounding tissue by overcoming the endothelial barrier. The duration (time interval) of the period of occlusion together with delivery of the regenerative cells is preset (prescribed) to produce these desired results. Additionally, this process serves to achieve a high concentration, retention, and distribution of the regenerative cells at the target site tissue, taking into account the particular site under treatment and limited by the capacity of tissue at the site to withstand cutoff of perfusion for the prescribed period without suffering tissue cell damage due to ischemia following the balloon inflation.

It will be apparent from the brief description thus far, and from the detailed description of working examples of methods of the invention to follow, that a process of transluminally delivering regenerative cells to a target site of tissue treatment through a blood vessel (or duct) in communication with the site, while occluding the vessel in proximity to the site for a period of limited duration but sufficiently long to increase pressure at the site and sufficiently short to avoid causing damage to tissue at the site, is independent of the specific regenerative cells utilized in the treatment. It is only essential that the regenerative cells—whether adult stem cells, autologous or allogeneic (allogenic), uncultured or fresh, unmanipulated or minimally manipulated, or engineered cells, for example—possess the capability to repair tissue of, and to rescue cells at risk (so-called antiapoptosis) in, the body part which is to receive treatment at the site. That is to say, the regenerative cells have a capacity to differentiate themselves to tissue of that body part.

The body part of interest may be, for example, an organ such as the heart, liver, brain, kidney or pancreas, or related vessels, glands, nerves or muscles, or other soft tissue or some other part. Although the methods of the invention can serve to assist in repair or rescue of cells of a particular body part—organ or otherwise—it is the delivered regenerative cells possessing the capacity to differentiate themselves that actually effect the repair or rescue. That is, over time while adhered or attached to the host tissue, such cells undergo morphogenesis; based on their differentiation capacity to establish the form, structure and function of tissue of the host organ or other body part by which they are retained. In essence, the delivered regenerative cells possess an ability to morph into the host tissue so as to possess all of the characteristics thereof. Successful treatment depends upon the level of concentration of the regenerative cells delivered at the selected site, and the level of retention of the cells by tissue at that site, as well as to overcome the endothelial barrier of the vessel and site walls, to ultimately achieve the differentiation induced by the microenvironment in which they are delivered.

In an exemplary method of the invention, intraluminal application of regenerative cells—in particular, autologous adult stem cells were used in this example—is performed in the above manner for delivery to the site of damaged tissue of the heart after a MI, to restore as closely as can be achieved the pumping performance of the myocardium. The method is guided by the principle of clinical practice of interventional cardiology that only those approaches that are both (a) relatively simple to perform, with little risk but of potentially significant benefit to the patient, and (b) highly cost effective, are appropriate to be routinely applied in everyday medicine. In a case of failing tissue of the myocardium, the regenerative cells were intraluminally injected via the coronary artery whose partial blockage led to the infarction, or into the corresponding coronary vein in a retrograde manner. It is hypothesized that the most effective way to deliver the cells to the infarct area is through the vascular tree of coronary arteries, arterioles and capillaries that supply the area. An occlusion balloon of an over-the-wire type catheter was inflated at a point closely proximate the site of the infarction, after the vessel had been re-canalized and the blood flow reconstituted.

While the blood flow is blocked, the regenerative cells are delivered to the targeted site by a single injection or repetitive injections through a lumen of the balloon catheter for the prescribed period of the occlusion. In the case of treatment of the heart, this period may be in a range of from 1 to 15 minutes, for example. Repetitive injections over a period of 15 minutes were used in one working example. The pressure increase at the site owing to this procedure enables large numbers of the injected regenerative cells to successfully attach outside the vessel within myocardial tissue adversely impacted by the infarct. Over a relatively short interval of time, aided by this increased pressure, at least an acceptable number of the retained regenerative cells is able to overcome the endothelial barrier and to migrate to a position beneath the damaged myocardial tissue. The resulting replaced or repaired myocardial tissue morphed from the regenerative cells aids in improved pumping action of the heart.

Rather than delivery though a blood vessel, the method may utilize application of regenerative cells through a duct that supports secretion flow from an organ or a related gland.

Accordingly, it is another aim of the invention to provide methods of transluminally delivering regenerative cells possessing a capacity to differentiate to host tissue via a vessel or duct (through a lumen of a catheter therein) to a targeted site of damaged tissue of an organ or other body part, while blocking blood or secretion flow, respectively, through the vessel or duct at or near the target site to increase local pressure at the site and thereby achieve concentration of large numbers of the delivered regenerative cells for considerable retention at the site.

### BRIEF DESCRIPTION OF THE DRAWINGS

The above and other aims, objectives, features, aspects and attendant advantages of the invention will become apparent to those skilled in the relevant art from the following detailed description of certain working examples of the methods of the invention, taken with reference to the accompanying figures of drawing, in which:

FIG. 1 is a transparent principally frontal view of a subject, illustrating exemplary locations of the subject's body from which autologous adult stem cells may be harvested, and of a manner of injecting the harvested stem cells into the subject's vascular system through a balloon catheter advanced to a selected or target site of tissue of the heart, for example;

FIG. 2 is a detailed view illustrating a portion of a vascular system into which a balloon catheter has been advanced, useful for explaining methods of the invention for injection of cells to a selected site, such as that of FIG. 1;

FIG. 3 is a transparent frontal view of a subject illustrating an exemplary apparatus installed in the subject's vascular system for injecting the harvested stem cells into the cerebral circulation of the subject, for example, useful for explaining another exemplary method of the invention;

FIGS. 3A and 3B are companion simplified views of syringes used in the course of performing the method described with respect to FIG. 3; and

FIGS. 3C and 3D are the same MRI image of hemispheres of the animal brain following certain experimental treatment according to the method described with respect to FIG. 3, with balloon occlusion (of carotid artery of right brain) and without balloon occlusion (of carotid artery of left brain) during injection; showing in FIG. 3C the intensity of, and in FIG. 3D, as well, a delineation of regions of greatest concentration of, stem cells in the right and left brain hemispheres;

FIG. 4 is a transparent frontal view of a subject illustrating an exemplary apparatus installed in the subject's body for applying harvested stem cells through a duct of the body, to a selected site of an organ such as the pancreas or liver, useful for explaining another exemplary method of the invention; and

FIGS. 5A and 5B are MRI images of an animal subject's right and left kidneys, respectively, showing the distributed population and concentration of stem cells following their intraluminal delivery thereto for situations of occlusion (right kidney, FIG. 5A) and non-occlusion (left kidney, FIG. 5B) of the respective renal arteries thereof.

### DETAILED DESCRIPTION OF EXEMPLARY METHODS OF THE INVENTION

According to the invention in its broadest aspect, each of the preferred methods begins with substantially the same basic process, namely, delivery of regenerative cells to a selected site of tissue in the body of a human or animal subject, by transluminal injection through a blood vessel in communication with the selected site, and temporarily concurrently blocking blood flow through the vessel at or slightly distal to the site for a prescribed period to increase pressure at the site. The duration of the period may be a single time interval or consecutive intervals of repetitive blocking and opening the lumen of the vessel, of sufficient overall length to allow the increased pressure to produce high levels of concentration and retention of the regenerative cells and migration thereof into host tissue at the selected site, but of shorter length than would cause damage to the host tissue. Blood flow through the vessel is restored upon expiration of the prescribed period(s) of blockage.

In the working examples of the methods of the invention to be described, the regenerative cells were harvested from the subject's own body, constituting autologous adult regenerative cells, and delivered to a targeted site of damaged tissue in the donor's body. Preferably, the harvesting and preparing of the adult regenerative cells, and injecting of the cells through a catheter installed in the lumen of a blood vessel or duct in communication with the targeted site, are performed in the same operative interventional procedure.

At a time prior to applicant's invention, it had been hypothesized by most researchers that adult stem cells are tissue specific. It was thought that a certain stem cell-like population exists in every organ and is capable of differentiation into this certain tissue, with exceptions to this rule regarding the heart and brain. Later, studies indicated an underestimated potential of these cells. It has been shown that murine and human neural stem cells (NSC) give rise to skeletal muscle after local injection (see, for example, Galli R et al., “Skeletal myogenic potential of human and mouse neural stem cells,” Nat Neurosci 2000; 3:986-991). Bone marrow stem cells have also been shown to replace and repair heart tissue (cardiomyocytes, endothelium, nerve structures, and vascular smooth muscle cells) after injection into lethally irradiated mice with a myocardial infarction (see Jackson K. A. et al., “Regeneration of ischemic cardiac muscle and vascular endothelium by adult stem cells,” J Clin Invest 2001; 107(11):11395-402). The tissue damage in general appears to transmit signals which direct multipotential stem cells to the site of destruction, and these precursors undergo a multi-step process of migration and differentiation at the organ site to replace damaged cells in form and function.

Experiments with cultured fetal cardiac myocytes or neonatal myocytes impose limitations owing to their heterologous nature and their possible induction of an immuno response necessitating an immune-suppressive therapy. Complications and risks associated with an immuno-suppressant therapy are an increased susceptibility to infection and the possible development of malignancies. In addition, it is speculated that only a few patients would be willing to undergo a long term immuno-suppressive therapy with all its negative side effects.

An alternative approach by Prockop suggests that marrow stromal cells act as stem cells for non hematopoietic tissue and are capable to differentiate into various types of cells including bone, muscle, fat, hyaline cartilage and myocytes (Prockop D. J. et al., “Marrow stromal cells for non hematopoietic stem tissues,” Science 1997; 276:71-74).

Some recent findings have stimulated interest in adult cardiomyocytes. A report in Nature describes the ability to inject adult bone marrow stem cells from transgenic mice into the border of infarcted myocardial tissue (Orlic D. et al., “Bone marrow cells regenerate infarcted myocardium,” Nature 2001; 410:701-5). According to this report, these adult stem cells are capable of differentiation into cardiomyoblasts, smooth muscle cells and endothelial cells after injection. The infarcted myocardium implied that the transplanted cells responded to signals from the injured myocardium which promoted their migration, proliferation and differentiation within the necrotic area of the ventricular wall.

One is then left to consider the most effective techniques to obtain adult stem cells. The classical technique to recover stem cells is a bone marrow tap. The bone marrow contains a wide variety of hematopoietic and mesenchymal stem cells in addition to the T-lymphocytes, macrophages, granulocytes and erythrocytes. By incubation with monoclonal antibodies specific for the respective cell lineages and by sorting and removing with a biomagnet after incubation with magnetic beads and cell sorting with FACS (fluoroscopy activated cell sorting), a highly enriched cell line of bone marrow derived stem cells can be isolated, cultured and grown.

Aside from the classical approach of a bone marrow tap, a more recent report states that cells from human adipose tissue contain a large degree of mesenchymal stem cells capable of differentiating into different tissues in the presence of lineage specific induction factors including differentiation into myogenic cells (see Zuk P. A. et al., “Multilineage cells from human adipose tissue: Implications for cell-based therapies,” Tiss Engin 2001; 7(2):211-28). The interesting approach in this research is that out of a lipoaspirate of 300 cm³ from the subcutaneous tissue, an average of 2−6×10⁸ cells can be recovered. Even if one assumes that after processing of this liposuction tissue and separation and isolation of the mesenchymal stem cells, only 10% of these stem cells might be left for culture, the remaining approximately 10⁷ (10 million) cells would be quite sufficient to be used for the intraluminal or transluminal transplantation methods described herein.

A benefit of this latter approach could be that culture and passaging of the stem cells might be avoided. This is of special importance, since in the early phases of myocardial infarction there is a high activity of inflammatory cytokines which promote adhesion, migration and proliferation of the stem cells. In addition, as long as there is no scar core tissue it is much easier for these cells to migrate into the whole area of myocardial infarction and resume the cardiac function.

Recently, embryonic stem cells have been the subject of intensive discussion, particularly their pluripotency to differentiate into a vast range of tissues and organs of the human body that are in need for repair. The discussion has included the potential use of such stem cells for replacement of insulin producing cells as well as embryonic stem cells that can differentiate into cells with structural and functional properties of cardiomyocytes. This is described in the August 2001 issue of the Journal of Clinical Investigation (Kehat I. et al., “Human embryonic stem cells can differentiate into myocytes with structural and functional properties of cardiomyocytes,” J Clin Invest 2001; 108: 407-14). Earlier, the proliferation of embryonic stem cells had been very elegantly described in principle by Field and co-workers (Klug M. G. et al., “Genetically selected cardiomyocytes from differentiating embryonic stem cells form stable intracardiac grafts,” J Clin Invest 1996; 98(1):216-24). In their attempt, the latter group succeeded in plating a cell line following a fusion gene consisting of the a—cardiac-myocyte-heavy-chain-promotor and the c-DNA encoding aminoglycosidephosphotransferase that was stably transfected into pluripotent embryonic stem cells. The resulting cell lines were differentiated in vitro and subjected to a G418 selection. By this means, the selected cardiomyocyte cultures were 99.6% pure and highly differentiated.

It is important to consider not only the engraftment of pluripotent embryonic stem cells into a failing organ, but also the possibility of resulting tumor formation. Therefore, the pluripotent embryonic stem cells need to be cultured in an undifferentiated status, transfected via electroporation and grown in differentiated cultures. The interesting approach in this work is the high yield of selected embryonic stem cell derived cardiomyocytes which, with simple genetic manipulation, can be used to produce pure cultures of cardiomyocytes. The authors assumed that this elective approach could be applicable to all stem cell derived cell lineages. It has also been reported by others that isolation of primate embryonic stem cells with cardiogenic differentiation is feasible (Thomson J. A. et al., “Isolation of a primate embryonic stem cell,” Proc Natl. Acad Sci USA 1995; 92:7844-48).

In addition, it has been reported that human cardiomyocytes can be generated from marrow stromal cells in vitro as well, but with a low yield of differentiated myocytes (Makino S. et at, “Cardiomyocytes can be generated from marrow stromal cells in vitro,” J Clin Invest 1999; 103:697-705). And as noted above, the 1997 Prockop report in Science describes another line of cardiomyocytes generated from marrow stromal cells in vitro. This cardiomyogenic cell line was derived from murine bone marrow stromal cells that were immortalized and treated with 5-azacytidine. By mechanically separating spontaneously beating cells, a cell line was isolated that resembled a structure of fetal ventricular cardiomyocytes expressing iso-forms of contractile protein genes such as alpha cardiomyocyte heavy chain, -light chain, a-actin, Nk×2.5-Csx, GATA-4, tef-1, MEF-2a and MEF-2D.

While these embryonic stem cells provide optimism for the future that cardiomyocytes derived from embryonic cells might fulfill the requirements of cells that can (a) be passaged indefinitely in culture, (b) be recovered from frozen stocks and are readily available if a patient with a myocardial infarction comes to the cath lab, (c) retain their differentiated cardiomyocyte phenotype and (d) maintain contractile activity with minimum or no immunogenity, further basic research is needed on those cells before they can be applied in the animal model. It is likely that a primate model of infarction and the transplantation of primate embryonic stem cell derived cardiomyocytes may be needed as the final proof of principle before a human study might be conducted.

Presently, for ethical, immunological and feasibility reasons the applicant herein submits that transplantation of regenerative cells comprising autologous adult stem cells (harvested from the same donor designated to receive them) is the most straightforward and practical approach to repair failing myocardium. Some working examples described herein promote invasion of ischemically injured cardiac tissue, for example, by stem cells that firmly attach and subsequently undergo differentiation into beating cardiomyocytes that are mechanically and electrically linked to adjacent healthy host myocardium. Adhesion of the injected stem cells and their migration beyond the endothelial barrier may be confirmed by observation after several days of frozen sections using light microscopy and, subsequently, electron microscopy. For evidence of the transition of stem cells into cardiomyocytes, markers are introduced into the stem cells before they are reinjected into the myocardial tissue to be repaired.

One approach might be to transplant male cells carrying the Y-chromosome into a female organism, but at least two factors weigh against this. It could lead to immunologic problems because of the different cell surfaces carried by the recipient and the donor (heterologous transplant), a potential reason that some studies are not able to show a successful heterologous cell transplantation. Even more importantly, a predominance of inflammatory cells exists at the site of myocardial injury, which leads to an immediate recognition of foreign cell surface proteins with consequent elimination of the cells. Use of autologous stem cells would not carry this immunologic risk of cell destruction, although some difficulty is encountered in prior introduction of genetic or protein markers into those cells.

To overcome this difficulty, a green fluorescence protein (GFP) is used as a marker, with introduction into the stem cell genome by liposomal gene transfer. Cells can then be identified after transplantation by fluorescence microscopy. As part of the procedure, stem cells are also marked by 3H-Thymidin, a radioactive labeled part of DNA. All stem cells undergoing DNA replication for mitosis will introduce 3H-Thymidin into their genome, and thus can be detected afterwards by gamma count. One limitation of this process is the fact that radioactivity (per volume) declines with each subsequent cell division (albeit initial total radioactivity stays constant). Nevertheless, this marker aids in developing a gross estimate of the amount of cells in a certain organ or tissue (e.g., heart, spleen, liver etc.).

Reference is now made to the accompanying figures of drawing, for describing exemplary methods of the invention by way of certain working examples. It is noteworthy that the figures are not intended to be to scale, nor to do more than serve as a visual aid to the description. In those figures representing the human body or body parts, certain body parts and apparatus components may be exaggerated relative to others for the sake of emphasis or clarity of the respective accompanying description. The regenerative cells—autologous adult stem cells in some of the working examples—are harvested in one of the known ways generally described above, such as by bone marrow tap or from adipose tissue, or by suctioning from lipoaspirate, for processing and possible cultivation.

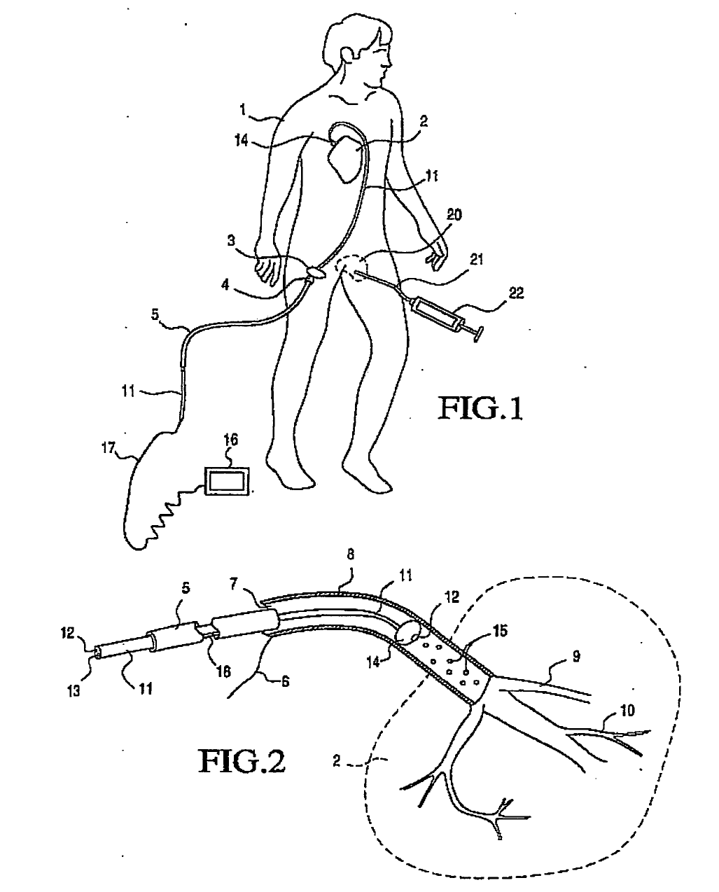

The aforementioned basic process of the invention is utilized in each of the methods of the invention, including that to be described with reference to FIG. 1. In an exemplary technique to obtain the regenerative cells, subcutaneous adipose tissue 20 is harvested from a subject under local anesthesia, here a patient 1, using a liposuction procedure. In this procedure, a hollow canule or needle 21 is introduced into the subcutaneous space through a small (approximately 1 cm) incision. Using gentle suction with a syringe 22 and moving the canule through the adipose compartment, fat tissue is mechanically disrupted, followed by injection of a solution of normal saline and a vasoconstrictor epinephrine, to recover (retrieve) about 300 cc of a lipoaspirate within the syringe. The lipoaspirate is processed immediately according to established methods, washed extensively in phosphate buffered saline (PBS) solution and digested with about 0.075% collagenase, or better, with a mixture of collagenase I, collagenase II, and a neutral protease, for example. The enzyme activity is neutralized with Dulbecco's modified eagle medium (DMEM) containing about 10% FBS (fetal bovine serum), for example, or just removed by washing. Following a centrifugation at about 1200 G for about 10 minutes, for example, a high density cellular pellet is obtained. Filtration through a filter of 100 micrometer pore size of appropriately tight Nylon mesh is then performed to remove cellular debris and unprocessed tissue structures. The cells are then ready to be injected into the patient's body.

Alternatively, the cells are further processed and incubated overnight in a control medium of DMEM, FBS, containing an antibiotic, antimycotic solution. After firm attachment of the stem cells to a plate, they are washed extensively with PBS solution to remove residual non-adherent red blood cells. Further cellular separation is performed by separation with monoclonal antibodies coated on magnetic beads, or cells are specifically modified to achieve a certain desired behavior or function.

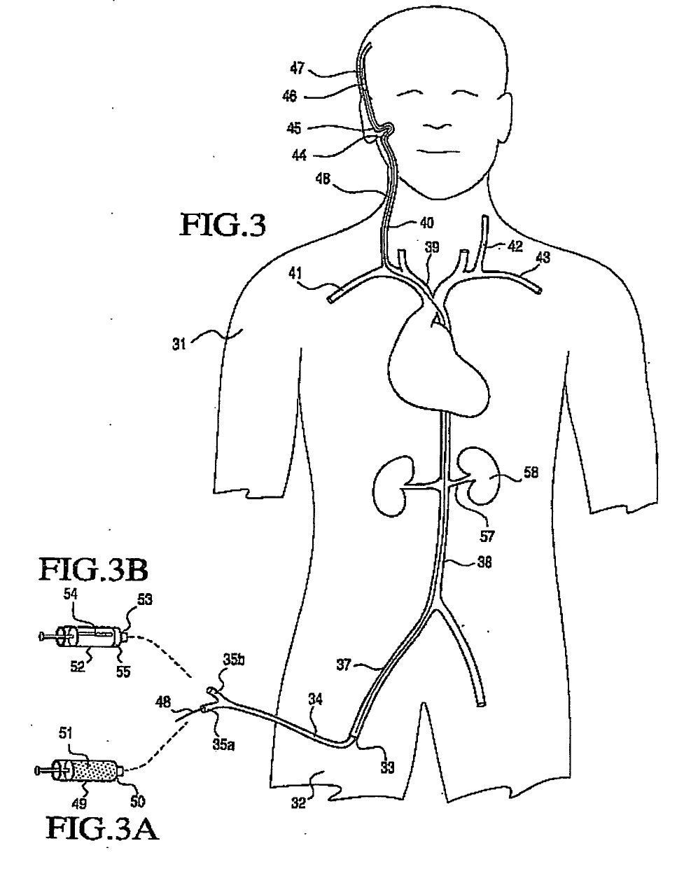

After a suitable quantity of the adult stem cells is recovered (e.g., 1× or 2×10⁷ to 10⁸), they are used (alone or as part of a regenerative cell mixture including multipotent stem cells with the adult autologous cells), by injecting them through a catheter installed in the lumen of a blood vessel or duct in communication with the selected site in the basic process of the invention. Preferably, the harvesting and preparing of the adult regenerative cells and their injection to the targeted site is performed in the same operative interventional procedure. Referring to both FIGS. 1 and 2, the autologous adult stem cells are injected in the donor subject 1 by transluminal application for delivery to the target (selected) internal site of interest. To that end, a balloon catheter 11 is introduced through vascular lumen 7 into the vascular system at the subject's groin 3 (for example) using an introducer 4, and through a guiding catheter 5 over a guide wire 18, and is advanced through the vessel 8 to the selected site. The advancement of the balloon catheter is continued until the outlet of the central lumen 12 at its distal end is positioned in close proximity to the selected site, as may be viewed by conventional fluoroscopy. Blood flow through the vascular system remains uninterrupted up to this time, notwithstanding the presence of the balloon catheter and the guiding apparatus in the vessel's lumen 7.

Preferably, the regenerative cell mixture is injected through the inner (central) lumen 12 of the balloon catheter 11 by activation of a motor driven constant speed injection syringe 16, via catheter 17 connected to the central lumen inlet at the proximal end of catheter 11. Alternatively, it may be injected into the balloon catheter's central lumen inlet manually using a syringe. Immediately prior to or at the moment the cells are to be injected, the balloon 14 of catheter 11 is inflated via the catheter's inflation lumen 13 to occlude the blood vessel. The balloon is positioned just slightly proximal to the distal end of the catheter, but in close proximity to the site targeted for treatment. The cells 15 are delivered to this site by transluminal application for the duration of the prescribed period of occlusion of the vessel 8.

The increase in the vascular segment's pressure distal to the occluding balloon addresses the fact that any matter within the blood vessel, including these regenerative cells, is separated from the tissue outside the vessel, including for example, that of a parenchymatous organ. The cardiovascular system is characterized by blood flow through the larger arteries into the smaller arteries, into the arterials, into the capillaries, and then into the venous system back into the systemic circulation. Normally, the regenerative cells delivered to the target site would be prevented from migrating to tissue outside the lumen of the vessel because of the barrier presented by the endothelial layer (endothelium), a layer of flat cells lining the blood vessels. In the basic process of the present invention, pressure at the target site is increased substantially by occlusion of the vessel produced by inflation of a single balloon at or proximal to the site, while injecting the regenerative cells, over the prescribed permissible period or duration of the occlusion.

Since the downstream capillary bed can only pass a limited number of corpuscular cells in a given time interval, the increasing number of cells that are injected into the blocked vascular segment are forced with increased pressure of injection to overcome the endothelial barrier. This is because these cells cannot “escape” by flowing back, because that escape route is blocked by the inflated balloon. This assures a considerably higher concentration of regenerative cells at the tissue outside the lumen of the blood vessel, and considerably greater levels of retention (adhesion) of the cells to tissue cells supported by the vessel lumen than are achievable without such occlusion. Additionally, since the increased pressure enables the regenerative cells to overcome the endothelial barrier, they are able to migrate into and proliferate at the tissue of the targeted site surrounding the vessel's lumen.

Use of a single balloon catheter 11 to selectively occlude the blood vessel and thereby prevent both antegrade blood flow (with consequent washout and dilution of the regenerative cells) and especially retrograde cell flow (backwash) at the target site, is preferred over the use of other devices or apparatus. The balloon catheter is a proven, frequently employed and reliable device for introduction into a blood vessel or a duct of the body in furtherance of a medical procedure. The device is commonly used in angioplasty and stent implant procedures, for example.

Thus, the basic process or method of the invention may be characterized as a method of optimizing regenerative cell retention at a tissue site in the body of a subject, comprising delivering the regenerative cells through a blood vessel in communication with the tissue site while occluding blood flow through the vessel for a prescribed period of sufficient duration to increase pressure at the site for enhanced concentration and retention of the regenerative cells at the tissue site, but of insufficient duration to cause damage to tissue at the site for lack of blood perfusion during the prescribed period.

In a first working example of use of the basic method of the invention to treat a MI patient with the patient's own recovered autologous adult regenerative cells, including stem cells, the cells are delivered to the target site of the infarct by intracoronary or transcoronary (transluminal) application (with reference again to FIGS. 1 and 2). Balloon catheter 11 is introduced into the cardiovascular system at the patient's groin 3 using introducer 4, and through guiding catheter 5 over guide wire 18, is advanced into the aorta 6 and thence the orifice 7 of coronary artery 8 of heart 2 at or in the immediate vicinity of the target site of tissue damage at the infarct. Prior to any attempt to place the balloon catheter and guidance apparatus through a blocked coronary artery, a re-canalization procedure is performed to open the artery, and typically such procedure is performed soon after hospitalization of the patient to restore flow of oxygenated blood to and beyond the infarct site. Blood is supplied through artery 8 and its distal branches 9 and 10. As noted above, the regenerative cells are preferably delivered through a central lumen 12 of the balloon catheter from motor driven constant speed injection syringe 16, which is coupled via connecting catheter 17 to lumen 12 at the proximal end of the balloon catheter. The regenerative cells exit from lumen 12 is at the distal end of catheter 11, near where balloon 14 of the catheter is located, slightly proximal to, but in any event in close proximity to the infarct site.

The regenerative cells 15 are delivered to this site by a simple injection over the duration of the prescribed period subject to limitations as described earlier herein. Alternatively, the cells may be delivered to the target site by repetitive injection steps over seconds to minutes each, with intervals of balloon deflation between each successive pair of injection steps to allow for intermittent blood flow, staged to occur between successive prescribed periods of relatively short duration to allow delivery of oxygenated blood to the site. Preferably, each of the prescribed periods of cell injection via the central lumen of the balloon catheter (coincident with blockage of the blood vessel) is set to have a duration in a range from less than about one minute to as much as about fifteen minutes, until the desired dosage of regenerative cells has been injected. The specific duration depends on the organ under treatment, location of the target site within that organ, its ischemic tolerance, and the underlying disease. A dosage in a range of approximately 10 million to 100 million regenerative cells at the targeted site should suffice, with the progression of time, to allow those cells to effect repair or replacement of the damaged tissue sufficiently to achieve some reasonable level of resumption of the myocardial pumping function (new or restored) at the site, subject to additional steps described below.

The endothelial ischemic damage attributable to the infarction allows white blood cells, especially granulocytes and macrophages, to attach via integrins to the endothelial layer of coronary artery 8 at the infarct site. The endothelial layer itself may be dissolved in places by the release of hydrogen peroxide (H₂O₂), which originates from the granulocytes. Consequently, gaps are produced in the endothelial layer that allow the invading regenerative cells to adhere to the endothelial integrins, and also to migrate through the gaps into the underlying tissue to repair damaged tissue or form new tissue that functions in the same manner as formerly provided by the tissue that was damaged by the MI. An ancillary factor, which acts as a chemo-attractant to the cells, further enables the regenerative cells to migrate into the organ (or other body part) tissue.

Nevertheless, these factors are inadequate to allow sufficient numbers of the regenerative cells delivered to the site and to provide force thereon to penetrate the endothelium and migrate into the underlying tissue and form new tissue cells and rescue other cells at risk. These deficiencies are more than overcome and the desirable elevation of pressure, regenerative cell concentration, retention and migration achieved by occlusion of the coronary artery for a prescribed period of limited duration, as described earlier herein. Preferred use of a balloon catheter 11 to assist in achieving these desirable results has also been explained herein. The balloon 14 of catheter 11 is inflated with biocompatible fluid through a separate lumen 13 of catheter 11 to occlude coronary artery 8 and its distal branches 9 and 10, thereby causing perfusion through the vasculature to cease distally of the occlusion. The balloon is inflated immediately before or at the time of injection of the stem cells through the inner lumen 12 of the catheter, and is maintained throughout the duration of blockage for a relatively short period of from about one to about fifteen minutes.

Dependent on the type and number of stem cells delivered, for treatment of the targeted MI site, the blockage is maintained for a relatively short period of time, such as described above, and in any event sufficient (subject to the consideration of period duration limitation to assure avoidance of any further coronary and/or myocardial tissue damage beyond that attributable to the MI) to enable a high concentration and considerable number of cell retention at the tissue of the target site, that will tend to produce a successful outcome. This repair will extend as well to any failing tissue that may result from the blockage itself. In the case of a slow infusion of the cells, especially if they are infused through a coronary venous vessel, the period of blockage is maintained longer by steady inflation of the balloon over the injection period (e.g., up to about 30 minutes, with intermittent periods of re-perfusion of about two to five minutes each to avoid additional tissue damage) for enhancement of contact with and adherence to tissue the target site. The balloon is then deflated, and the balloon catheter is removed from the patient following this procedure.

Previously reported studies have invariably employed a surgical approach for the application of the cells to be transplanted. Even if five to ten sites of injection are performed with small needles, the complete inner, medial and outer layers of the myocardium are never covered. The examples described herein use the natural distribution tree of the arterioles and the capillaries which is a more elegant solution, provided that (1) interventional cardiology means can restore blood flow into the infarct area again, and (2) the duration of blockage of the coronary artery is sufficient for the injected regenerative cells to overcome the endothelial barrier.

In clinical practice there is a 96% success rate with interventional cardiology to restore blood flow following an acute myocardial infarction resulting from blockage of a coronary artery attributable to disease. The experience that venously injected stem cells can be found in the myocardium, and the knowledge that in an acute myocardial infarction the endothelial barrier is considerably damaged (partly due to the H₂O₂ release of adhering neutrophilic cells), lead to the conclusion that a local injection into the infarct area with an occlusive balloon to prevent a washout of the cells is the most desirable approach.

The applicant herein has performed studies in the past with a technique called ‘BOILER’-lysis, in situations where older venous bypass grafts are occluded by a thrombus that has grown over a prolonged period of time. It was observed that an acute injection of a thrombolytic agent rarely dissolved these old thrombi. But after an over-the-wire balloon catheter was inserted into the occluded graft, a prolonged application of a thrombolytic substance such as urokinase was successful in achieving thrombolysis. The agent is injected at the tip of the balloon catheter, and is forced antegradely into the thrombus. The inflated balloon prevents a washout by the normal coronary circulation and allows the injection by a motor pump at a defined volume per time. For regenerative cell therapy and capacity for repair as performed by the exemplary methods described herein, injection over a total period in a range from about one to about thirty minutes is feasible (with intermittent re-perfusion at the longer periods of the range) and gives the cells sufficient contact time for retention by the damaged myocardium.

Only a portion of the myocardial cells that had been ischemic following an MI will survive, depending on the interval of time that passes before restoration of blood flow is achieved. A typical procedure is to perform a balloon angioplasty of the blocked artery, followed by implanting a stent at the site of the lesion. But even in the case of optimal treatment some 40% of the affected cells are likely to die. The transluminal injection of stem cells according to the method of the invention should be performed as soon as possible after re-canalization, so as to take advantage of the profusion of inflammatory cytokines that accompanies an infarction, which promote the adhesion, migration and proliferation of the regenerative cells in the tissue. Occlusion of blood flow is desirable here to take advantage of this earlier inflammation.

The methods of the invention are not limited to a capability for cellular repair (i.e., by regenerative cell differentiation to characteristics and function corresponding to host cells) of damaged myocardial tissue. Other body organs having damaged tissue for which similarly successful treatment may be achieved include, for example, the pancreas, the liver, and the kidneys. The pancreas has a duct (the ductus Wirsungii) through which pancreatic enzymes are delivered into the intestines, and which can be accessed in a retrograde manner by endoscopic retrograde choledocho-pancreaticography (ERCP). Failing tissue in the case of a diabetic patient means that the pancreatic cells therein no longer produce sufficient insulin for the patient's needs. By means of the visual guidance through a small fiberglass instrument a small balloon catheter may be introduced into this duct, and the balloon inflated to occlude the duct during delivery of regenerative cells through the catheter's inner lumen to the site of the damaged tissue, so as to prevent the injected cells from being washed out into the intestines, and thereby to enhance large scale concentration, retention and penetration of the cells to the target tissue.

The basic method of the invention may also be used to provide a capacity for repair of damaged tissue of the liver, through the bile duct system. Here also, it is important to overcome the barrier of the normal bile duct with pressure that is generated as the balloon is inflated while the cells are slowly injected. The pressure distally of the injection site increases as more and more cells are injected. Failing tissue in the kidney(s) may be treated with regenerative cells having a capability to repair, by employing the basic method of the invention with appropriate attention to limitations such as duration of vessel blockage, for example, in the case of renal failure. Here, an inflatable balloon catheter of appropriate balloon size of 4 to 8 mm and short balloon of 9 to 15 mm is placed in the renal artery. Inflation of the balloon to block blood flow and injection of regenerative cells is performed in substantially the same method as described above for the heart. Afterward, the balloon is deflated and the balloon catheter is removed from the subject's body.

The methods of the invention may be applied also to the brain in the case of a patient having suffered cerebral damage from, for example, an infarction or other neurological deficit. Previous studies have indicated that regenerative cells have the capacity to replace neural cells in the brain and, therefore, overturn the consequences of an acute or chronic vascular stroke or damage. In this case, the injection catheter is advanced to the site of the damaged tissue through an appropriate arterial path into the applicable region of the patient's brain. Blockage of blood flow in this case would be considerably more limited in duration than in the case of treatment of an MI, for example.

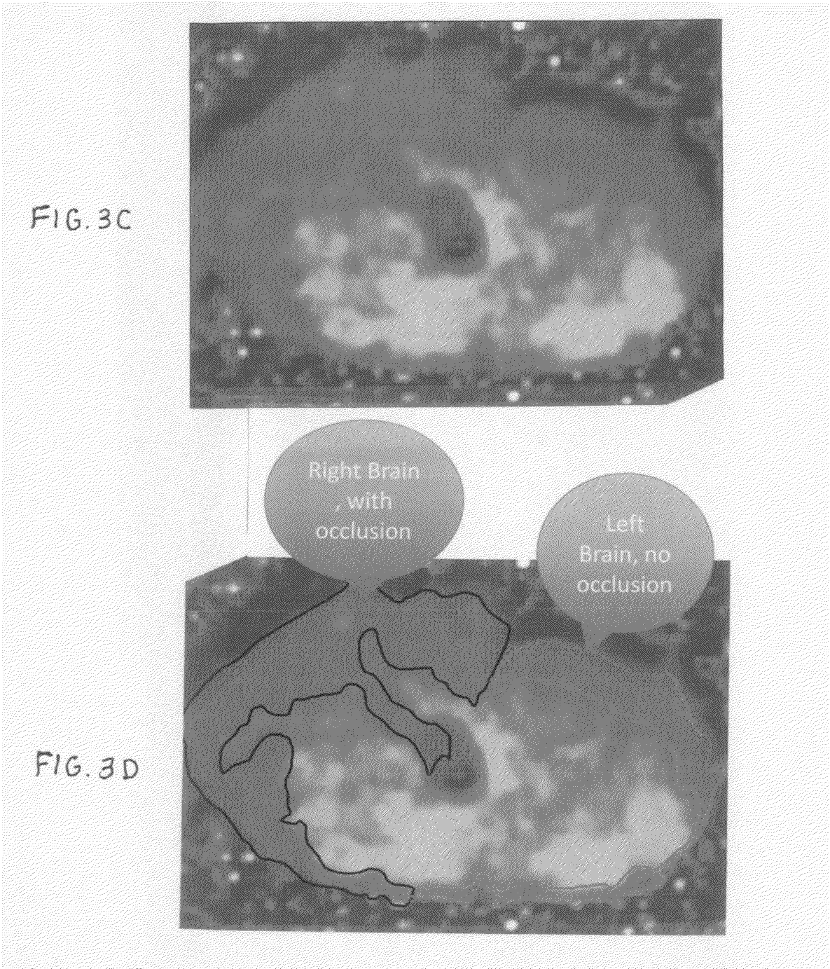

FIG. 3 is an illustration of a patient 31 undergoing treatment of the brain, utilizing the basic method of the invention, appropriately modified to accommodate issues relevant to that part of the central nervous system, for delivery of regenerative cells through a balloon-guided catheter to the anterior cerebral circulation of the patient. In a working example of the method, an introducer sheath 33 of appropriate size, such as, for example, 5-7 French, is advanced through the right groin 32. Then a balloon guided double lumen catheter 34 is advanced through introducer sheath 33 and over a small guide wire 48 placed in the artery of interest. Guide wire 48 has a diameter in a range of about 0.014 to about 0.018 inches, and a flexible distal tip to render it bendable so as to direct the guide wire through the vessel to the vicinity or locality of the selected target site. The proximal end of guide wire 48 is left to project from opening 35a of catheter 34. A side branch opening 35b of catheter 34 is operatively coupled through an inflation lumen of the catheter for selective inflation and deflation of its balloon 46.

Initially, guide wire 48 is advanced through the central lumen of catheter 34. The catheter may then be maneuvered to the selected site by gliding it over the guide wire through iliac artery 37, abdominal and thoracic aorta 38, through the aortic arch 39, and into the right carotid artery 40 beyond the branching off of the vessels 41 for the right arm. As an alternative, guide wire 48 and catheter 34 may be advanced to a location in the left carotid artery 42. The left carotid artery either originates after the branch-off of the left subclavian artery 43, or directly from the aortic arch 39 where the left subclavian artery originates from a separate orifice in the aortic arch.

After advancing guide wire 48 through the common carotid artery into the right internal carotid artery 40 and into the proximal circulation of the Circulus Willisi 44, the anterior cerebral artery 45 is encountered at its origination. After the catheter 34 has been advanced so that its distal tip 47 and balloon 46 are positioned in the anterior cerebral artery 45, with the catheter tip 47 located at the target site to which the harvested autologous adult regenerative cells, including stem cells, are to be delivered, guide wire 48 is removed. The opening 35a of the same lumen that had been used for the guide wire is now available for injecting regenerative cells for delivery to that site.

Toward that end, and with reference now also to FIGS. 3A and 3B, the conus 50 of a syringe 49 (FIG. 3A) is connected to port 35a of catheter 34, and the conus 53 of another syringe 52 (FIG. 3B) is connected to the inflation port 35b of catheter 34. Port 35b operates through the inflation lumen for balloon 46 of catheter 34. Syringe 52 is of small size and includes a pressure gauge 55 to measure the applied pressure as the fluid 54 within the syringe is expelled into port 35b to inflate balloon 46 to a low pressure of, for example, about 0.5 to about 4 atm. This pressure is sufficient to tightly seal the vessel (anterior cerebral artery 45) at the location of the balloon. To assist in recognizing a possible rupture of balloon 46, the fluid 54 in syringe 52 is preferably about a 50/50 mixture of saline and contrast dye. Balloon 46 may be deflated at the completion of the procedure or in the event of an emergency by withdrawing the fluid 54 back into syringe 52.

While anterior cerebral artery 45 is tightly sealed toward its proximal end 44, stem cells 51 within syringe 49 are ejected from conus 50 into port 35a of the catheter. The regenerative cells travel through the central lumen of catheter 34 formerly occupied by guide wire 48 and exit the lumen at the site of catheter tip 47. The regenerative cells are thus delivered for entry into the cerebral circulation at that site.

As noted earlier herein, the very brief period of less than one minute, and preferably of five to fifteen seconds, of limited blood supply during blockage of blood flow through the anterior cerebral artery 45 by inflated balloon 46 is of sufficient duration for the regenerative cells to overcome the endothelial barrier but not of such long duration as to cause injury to the brain cells in the region normally nourished by oxygenated blood flow.

A method according to the invention was used in another experimental working example of treating a brain. Here, porcine adipose derived mesenchymal stem cells were labeled with Micrometer-sized Paramagnetic Iron Oxide (MPIO) particles according to published methods. Cell delivery was performed via a commercially worldwide distributed standard over-the-wire balloon catheter (3 mm balloon diameter and 9 mm balloon length, obtained from, for example, Boston Scientific, Inc.).

The catheter was placed into the right internal carotid artery using a 0.014 floppy guide wire under fluoroscopic guidance through a 6F guiding catheter by standard cardiovascular intervention techniques. After placing the floppy guide wire in the internal carotid artery, the balloon catheter was advanced over the guide wire. After correct placement of the balloon catheter in the internal carotid artery, the wire was removed from the catheter's central lumen, and the vessel was occluded by balloon inflation at 4 atmospheres. Immediately after occlusion, 5×10⁶ labeled stem cells (acquired as stated above) in 5 ml isotonic saline were injected over a prescribed period (time interval) of five seconds duration. The balloon was deflated immediately after cell delivery, and blood flow was restored as verified by contrast dye injection through the guiding catheter.

The period of occlusion was expressly prescribed to be long enough to increase pressure sufficiently in the vicinity of the selected tissue site of stem cell delivery so as to increase the concentration and retention of the injected stem cells at the site, but not of a length that could result in tissue damage at the site or anywhere upstream of the occlusion point. A period in the range from three to ten seconds would serve the intended purpose without adverse outcome. The guiding catheter and balloon catheter were then withdrawn into the aorta.

Then the guiding catheter was placed into the left carotid artery, and the cell delivery procedure was repeated identically as described above with the balloon catheter advanced therein, but with the sole exception that the balloon was not inflated either prior to, during, or after cell delivery. The balloon catheter was withdrawn after delivery, and blood flow was verified by contrast dye injection through the guiding catheter, and the guiding catheter and balloon catheter were withdrawn into the aorta.

FIGS. 3C and 3D are the same MRI image of hemispheres of the brain following certain experimental treatment, without delineation and with delineation, respectively, taken immediately after performance of the above experiments. The FIG. 3C MRI image of the brain shows the right brain on the left side of the image and the left brain on the right side of the image. The T2 star map weighted MRI analysis is specifically dedicated to detect iron within the labeled cells, and accordingly is directed to assessing the number of cells that correlate with the amount of iron detected by MRI. The presence and number of MPIO particle labeled stem cells injected into the right and left carotid arteries and retained in the right and left hemispheres, respectively, where the injection modes were with balloon occlusion (right hemisphere) and without balloon occlusion (left hemisphere), is clearly observable in the Figure by the areas of deepest red color (indicative of presence of high iron from the concentration, retention and number of the injected labeled regenerative cells), whereas areas of yellow color are indicative of low iron. At twelve different points of the respective hemisphere a measurement of the signal intensity was performed as an indicator of the presence of the MPIO-labeled stem cells therein.

The data (as indicated by the Figure) show that a significantly higher concentration, distribution and number of cells was found in the right brain (left side of FIG. 3C), where the cells were delivered while the right carotid artery was occluded by the inflated balloon, compared to the left hemisphere (right side of FIG. 3C), where the cells were injected without balloon inflation and thus no occlusion of the left carotid artery, but under otherwise identical conditions. FIG. 3D shows the high intensity iron region of both hemispheres, just as in FIG. 3C, but with the respective regions delineated with a black line on the right brain, and delineated with a green line on the left brain. The data indicates that the occlusion of the right carotid artery during regenerative cell delivery to the target site resulted in a cell retention area on the right brain 2.37 times larger than the cell retention area on the left brain, where regenerative cell delivery was performed without occlusion of the left carotid artery before, during or after the cell delivery.

Applicant herein and others have shown that mesenchymal stem cells (such as those used in this working example) demonstrably have the capability to repair damaged tissue. Transluminal delivery of an adequate quantity and retention of the cells at the site of the tissue damage, due to the increased pressure owing to arterial occlusion coincident with cell injection for the prescribed period(s) according to the method(s) of the present invention, allow greater numbers, concentration and retention of such cells to be delivered to a targeted site, having the capacity to morph into healthy brain cells simulating the characteristics and function of the damaged tissue cells prior to their injury.

A working example of a method of the invention used in treating the kidney(s) with stem cells will now be described, with reference again to FIGS. 3, 3A and 3B. Here also, porcine adipose harvested mesenchymal stem cells with MPIO-labeling were used in the working example for treating a kidney. The mesenchymal stem cells were introduced by transluminal application through a balloon catheter navigated over a guide wire in the animal patient's right groin into the iliac artery 37, the abdominal aorta 38, the applicable renal artery 57, and the respective kidney 58. The guiding catheter was slightly withdrawn backwards into the abdominal aorta, and the orifice of the guiding catheter was placed in the right renal artery under fluoroscopic guidance. The correct position was verified by cine-angiography under contrast agent injection. The 0.014″ floppy guiding catheter was placed in the middle of the renal artery, and a 6 mm diameter over-the-wire balloon catheter of 10 mm length was placed one centimeter distal of the orifice of the artery. Then the floppy guidewire was removed to make the central catheter lumen usable for injection of the stem cells.

The balloon was inflated for a period of only five seconds despite longer inflation periods of up to ten minutes being feasible based on the ischemic tolerance of the kidney tissue. Immediately after balloon inflation and blockage of blood flow, preventing retrograde flow of the injected cells, five million cells suspended in 5 milliliters of saline solution were injected transluminally through the catheter in the right renal artery, over the indicated period of five seconds. This latter period is prescribed for the same purpose and intended result as described above for the brain working example (except that the selected site here is in the region of the respective kidney). The balloon was then deflated, and both the guiding catheter and balloon catheter were withdrawn into the abdominal aorta after the cell injection. Subsequently, the guiding catheter and balloon catheter were placed at the left renal artery and the procedure was repeated as described above, with the sole exception that the balloon was not inflated during the cell injection. So the right kidney received cell delivery with balloon occlusion, and the left kidney received cell delivery without balloon occlusion.

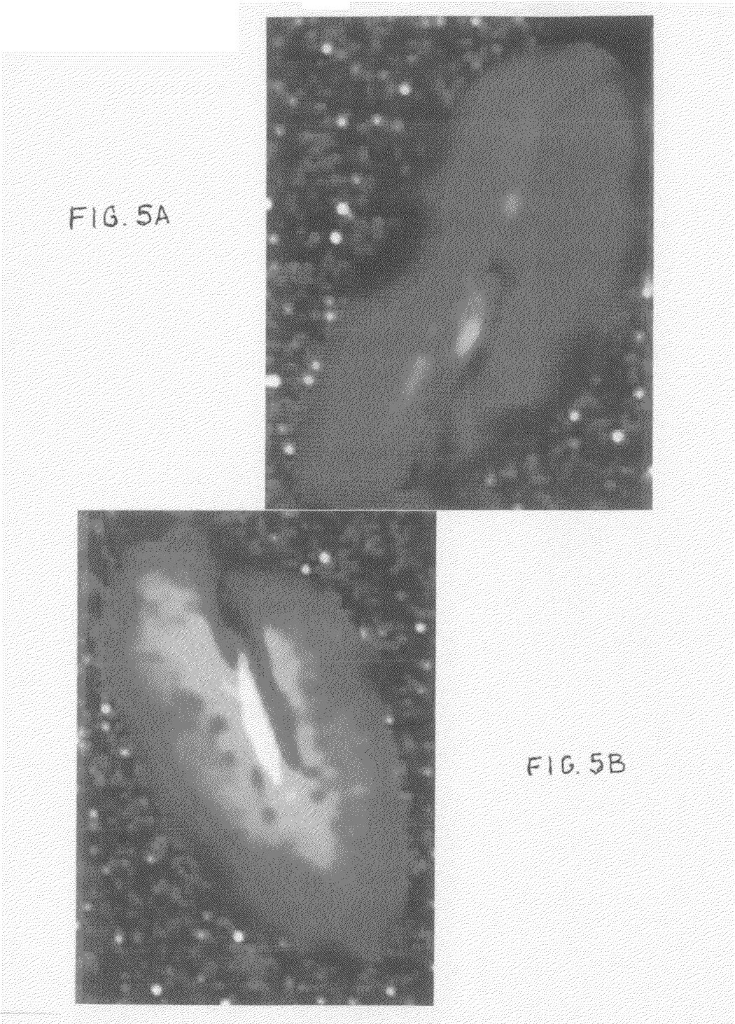

FIGS. 5A and 5B are MRI images of an animal subject's right and left kidneys, respectively, illustrating the distributed population of stem cells introduced therein for situations of occlusion and non-occlusion of the respective renal arteries following intraluminal delivery of stem cells to those sites, according to the working example described above. Here again, areas indicating highest intensities of red color are representative of high iron, and thus highest concentration, retention and number of the MPIO-labeled stem cells on the kidney, while areas of yellow color are representative of low levels of iron. Evaluation of the MRI images was performed in the same way as with the brain, indicating a significantly higher distribution, concentration, retention and number of MPIO-labeled stem cells retained in the right kidney as reflected by the higher iron content in the measurement for cells injected into the right renal artery under balloon inflation, compared to the left kidney where the cell injection was performed without renal artery occlusion.

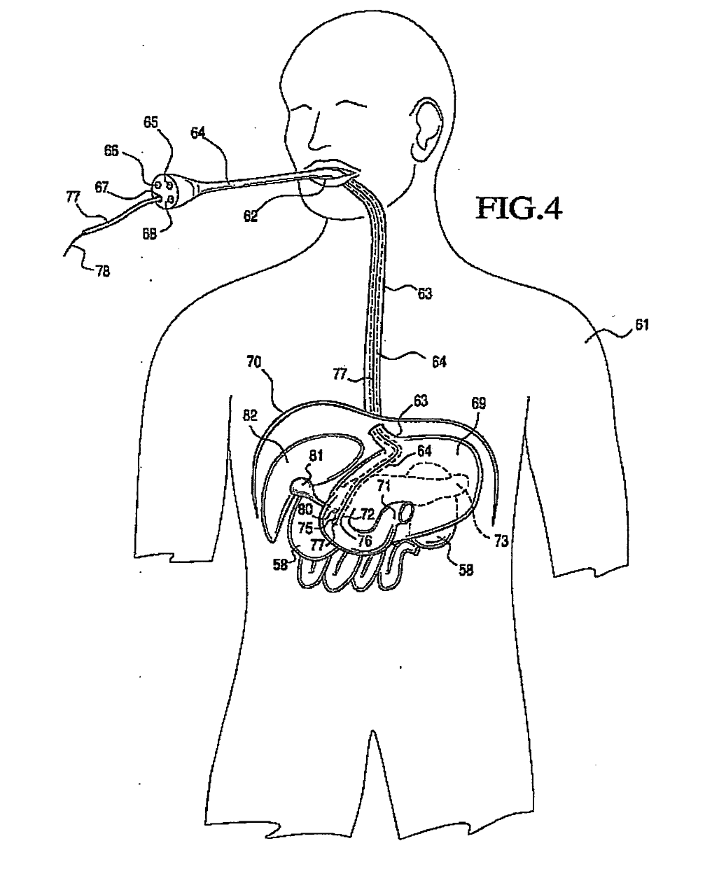

FIG. 4 is a diagram useful to describe another working example of a method for delivery of regenerative cells; however, in this example, the delivery is through a natural duct in a patient 61. In this exemplary procedure, an endoscope 64 is advanced through the mouth 62 and esophagus 63 of the patient. The endoscope 64 is flexible, and is designed and implemented with a plurality of channels including, in this illustrative example, a visualization and fiber optics channel 65, flushing channel 66, side port open channel 67, and working channel 68. The distal tip 75 of endoscope 64 is readily bendable to allow the endoscope to be advanced through a tortuous path. During the procedure the patient may be give a local anesthetic to prevent gagging.

The endoscope 64 is advanced from the esophagus 63 through the diaphragm 70, into and through the stomach 69, and further until its distal tip is located in the duodenum 71.

If the pancreas is the organ whose tissue is to be treated by stem cells capable thereof through differentiation to tissue of the organ, the location of the distal tip should be such that a side port 72 of the endoscope adjacent its distal tip is aligned for entry into the ductus Wirsungii 76, which supports the internal structure of the pancreas 73 with all its side branches. Proper alignment may be verified through the visualization and fiber optics channel 65 of endoscope 64. Then, a small balloon guided catheter 77 (e.g., 2.7 French outer diameter) is advanced over a guide wire 78 threaded through the side port open channel 67 and out of the side port 72 into the ductus Wirsungii.

Stem cells are delivered and the balloon is inflated by the use of syringes in a method similar to that described with respect to FIGS. 3A and 3B. The distal tip of the catheter is advanced through channel 67 of the endoscope 64 and out of the side port 72 to the site of the pancreatic tissue to be treated. The catheter's balloon is then inflated through the inflation lumen of the catheter to occlude the Wirsungii duct while stem cells are introduced into the pancreatic tissue through the central lumen of the catheter which is now open following removal of the guide wire. By proper positioning of the catheter's distal tip at the target site of the pancreatic tissue to be treated, the regenerative cells are delivered into the locale of that site. Occlusion of the duct precludes the stem cells from washing out into the intestines, so as to enhance penetration of the cells to the target tissue and large scale adhesions.

If the patient's liver 82 is the organ whose tissue is to be treated by delivery of stem cells through a natural duct, the distal tip 75 of endoscope 64 is positioned in the duodenum 71 such that its side port 72 is aligned for entry into the common biliary duct 80, which supports the liver 82 and the gall bladder 81. As an alternative, the side branch of the bile duct may be used. The guide wire and balloon catheter are fed through channel 67 and out of side port 72 of the endoscope, into the duct. The distal tip of the catheter is positioned at the target site of the liver tissue, the guide wire is removed, and the catheter's balloon is inflated to occlude the biliary duct during the introduction of stem cells. The cells are injected through the central lumen of the catheter for adhesion to and engraftment at the target site of the liver tissue.

The methods of the invention may also be utilized to treat other body parts, such as to rescue tissue resident cells at risk of apoptosis, to form new blood vessels, to exhibit an anti-inflammatory effect, and locally to act as immune-modulation. Thus, target sites may be not only of organs such as heart, brain, kidney, liver, pancreas, bowels, and lungs, but also of tissue such as muscle, bone, cartilage, and other soft tissue within the body of a subject as long as it is supported and supplied by the vascular circulation or, in the case of some body parts, by ductile flow.

Although certain methods and procedures have been described with particularity herein, the scope of coverage is not limited thereto. Rather, the scope of the invention is to be determined from the appended claims as construed according to applicable law.

What is claimed is:

1. A method for delivering regenerative cells to a targeted site of tissue in the body of a human or animal subject, including the steps of:
injecting the regenerative cells through the lumen of a blood vessel in communication with the targeted site for delivery thereto;
during the delivery temporarily occluding the blood vessel at the targeted site for at least one period having a prescribed duration shorter than would cause damage to the tissue at the site for lack of oxygenated blood flow, but of sufficient length to produce increased pressure distal to the occlusion whereby to enhance levels of concentration and retention of the regenerative cells at tissue of the targeted site, and
restoring blood flow through the vessel upon expiration of the prescribed duration of the period.

2. The method of claim 1, including setting the prescribed duration of the period to be of sufficient length to produce said increased pressure whereby to also enable the retained regenerative cells to migrate into tissue at the targeted site and to overcome an endothelial barrier in the tissue, while maintaining the duration to be shorter than would cause damage to the tissue.

3. The method of claim 1, including selecting regenerative cells having a capability of differentiation to ultimately assume characteristics of tissue at the targeted site, for the delivery.

4. The method of claim 3, including selecting autologous adult regenerative cells for the delivery.

5. The method of claim 1, including inserting a balloon catheter through the blood vessel lumen for delivery of the regenerative cells via a lumen of the catheter, and inflating the balloon for the occlusion of the blood vessel for the prescribed period.

6. The method of claim 5, including introducing a guide wire through the blood vessel to the targeted site, and thereafter advancing the catheter over the guide wire until the distal end of the catheter reaches a selected point in close proximity to the targeted site for delivery of the regenerative cells thereto.

7. The method of claim 1, including harvesting adult regenerative cells from the subject's own body, for delivery to the selected site.

8. The method of claim 1, including selecting for the delivery adult regenerative cells harvested from a location in the subject's own body predetermined to improve the likelihood of differentiation of the harvested cells to exhibit the same characteristics as tissue at the targeted site.

9. The method of claim 8, including harvesting and preparing the adult regenerative cells and injecting the cells to the targeted site in the same operative interventional procedure.

10. The method of claim 1, including temporarily occluding the blood vessel at the targeted site for successive periods each corresponding in duration to the duration of said at least one period during the delivery of the regenerative cells, and interrupting the successive periods of occlusion to restore blood flow between the successive periods while concomitantly interrupting delivery of regenerative cells.

11. A method of optimizing regenerative cell retention at a tissue site in the body of a subject, comprising delivering the regenerative cells through a blood vessel in communication with the tissue site while occluding blood flow through the vessel at the site for a prescribed period of sufficient duration to increase pressure at the site for enhanced concentration and retention of the regenerative cells at the tissue site, but of insufficient duration to cause damage to healthy tissue at the site for lack of blood perfusion during said period.

12. The method of claim 11, wherein said prescribed period of sufficient duration increases pressure at the site sufficiently for retained regenerative cells to overcome an endothelial barrier in host tissue at the site.

13. The method of claim 11, including deriving the regenerative cells from the body of the subject, as autologous adult multipotent cells.

14. The method of claim 11, including advancing a balloon catheter through the blood vessel to position the distal end of the catheter proximate the tissue site to allow delivery of the regenerative cells through a lumen thereof to the site and concurrent occlusion of the vessel by inflation of the balloon of the catheter through an inflation lumen thereof.

15. A method of treating damaged tissue at a targeted site in the body of a subject, comprising harvesting regenerative cells from the body of the subject, said regenerative cells having a capacity to differentiate to assume characteristics of host tissue when in contact therewith, and delivering them intraluminally to the targeted site through a lumen of a balloon catheter positioned in a blood vessel of the subject in communication with the site while occluding the vessel so as to increase pressure at the targeted site and thereby increase concentration and retention of the delivered regenerative cells at the site, and thereafter opening the blood vessel to restore perfusion therethrough before a lack of oxygenated blood causes damage to healthy tissue at the site.

16. The method of claim 15, including selecting the regenerative cells for their capacity to repair damaged tissue of the type at said targeted site by virtue of differentiation to the tissue characteristics while in contact therewith for a sufficient interval of time.

17. The method of claim 15, including limiting occlusion of the vessel to a period of duration sufficiently long to produce said increased pressure, but short enough to avoid damage to healthy tissue at the targeted site.

18. The method of claim 17, including occluding the blood vessel at the targeted site for successive periods each corresponding in duration to the duration of said period during the delivery of the regenerative cells, and interrupting the successive periods of occlusion to restore blood flow between the successive periods while concurrently interrupting delivery of regenerative cells.
